(19) United States
(12) Patent Application Publication
(10) Pub. No.: US 2020/0246050 A1
(43) Pub. Date: Aug. 6, 2020

(54) LOCKING SCREW AND PLATE SYSTEM USEFUL IN ORTHOPEDIC APPLICATIONS

(71) Applicant: IMEX Veterinary, Inc., Longview, TX (US)

(72) Inventors: Romano Matthys-Mark, Horgen (CH); T. Hall Griffin, Longview, TX (US)

(21) Appl. No.: 16/777,953

(22) Filed: Jan. 31, 2020

Related U.S. Application Data
(60) Provisional application No. 62/799,562, filed on Jan. 31, 2019.

(51) Int. Cl. A61B 17/80; A61B 17/86; A61B 17/88

(52) U.S. Cl. CPC A61B 17/8057; A61B 17/8615; A61B 17/8888; A61B 17/8625

(57) ABSTRACT

An orthopedic screw and plate system in which the screw has a single male thread profile disposed on its shaft. The thread profile is uniform and unitary. The plate provides plate holes. A female thread profile is disposed on the interior of the plate holes, and extends through the thickness of the plate. The female thread profile is configured for matching threaded engagement with the male thread profile on the shaft of the screw, such that when the screw is threadably received through the plate hole and into a bone, threaded engagement between the screw and the plate locks the plate in a desired position relative to the bone. Methods of fixation are also described using the disclosed screw and plate system.

### PRIORITY CLAIM

This application claims the benefit of and priority to co-pending, commonly-owned and commonly-invented U.S. Provisional Patent Application Ser. No. 62/799,562 filed Jan. 31, 2019. The disclosure of 62/799,562 is referred to herein as the “Provisional Disclosure”. The entire disclosure of Provisional Disclosure is incorporated herein by reference as if fully set forth herein.

### FIELD OF THE DISCLOSURE

This disclosure is directed generally to orthopedic fixation technology, and more specifically to a locking screw and plate system. The system combines to provide firm bone fixation and angular stability of the screw in relation to its corresponding locking plate.

### BACKGROUND OF THE DISCLOSED TECHNOLOGY

The field of orthopedics includes the treatment of disorders such as, without limitation, bone fractures, deformities, tumors, luxations, and arthritis. It is well understood in the treatment of such orthopedic disorders that it is advantageous to secure and stabilize bones, bone fragments and/or joints in desired alignment and to maintain such alignment and stability during healing. Orthopedic systems for such alignment and stabilization include: bone plates and screws, intramedullary pins and nails, external skeletal fixators, orthopedic wire and more.

Historically, cooperating locking bone screw and plate systems have proven useful in treatment of orthopedic disorders. In particular, successful conventional locking bone screw and plate systems have provided an angle-stable relationship between the plate, screw and bone, in that properly-fitted interlock between screw and plate causes the screw to maintain a constant and stable angular position of the screw in relation to the plate.

Conventional cooperating locking plate and screw systems typically achieve angle-stable relationship of the screw and plate by one or more of the following features:

1. A second or third thread profile on the screw with the primary thread engaging the bone and a secondary or tertiary thread engaging the plate.

2. A third or fourth component in addition to the locking plate and screw (such as locking rings or nuts) to provide or enhance locking of the plate and screw.

3. A tapered relationship between the screw and bone plate—such as Morse Taper (smooth), or tapered threading potentially included in combination with feature no. 1 above.

Such conventional locking screw and plate combinations include those disclosed in U.S. Pat. No. 7,799,062 to Crozet. Crozet discloses at least two separate and different thread profiles on the screw—one for bone engagement and another for locking plate engagement. The screw is also disclosed recessed into the locking plate.

It would be advantageous to improve on such conventional systems by, for example, simplifying manufacture, making the systems more compact, and eliminating additional components, while still maintaining firm bone fixation and angle stability.

### SUMMARY OF DISCLOSED TECHNOLOGY AND TECHNICAL ADVANTAGES

These and other drawbacks in the prior art are addressed by a locking screw and plate system in which the screw has a single male thread profile disposed on its shaft. The thread profile is uniform and unitary. The plate provides plate holes. A female thread profile is disposed on the interior of the plate holes, and extends through the thickness of the plate. The female thread profile is configured for matching threaded engagement with the male thread profile on the shaft of the screw, such that when the screw is threadably received through the plate hole and into a bone, threaded engagement between the screw and the plate locks the plate in a desired position relative to the bone. Methods of fixation are also described herein for using the disclosed screw and plate system. As the screw is rotationally advanced during fixation, the unitary screw threads engage the plate and at the same time engage the bone, providing angular stability between the screw and plate. Definitive angle-stable locking is thereby provided.

It is therefore a technical advantage of the disclosed locking screw and plate system to simplify manufacture of the screw. The preferred unitary, uniform thread profile on the screw obviates the need for secondary thread profiles or tapering threads such as seen in the prior art.

A further technical advantage of the disclosed locking screw and plate system is to obviate the need for special mating areas in the plate (such as the recessed hole in the plate in Crozet). This simplifies manufacture. This also yields a plate from which the screw may enter from either side during fixation. The surgeon is thus not limited as to “which way up” to deploy the plate, enhancing convenience, or bringing plate shapes and geometries into play whose orientation may be selected according to which side of the plate is facing the bone surface.

A further technical advantage of the disclosed locking screw and plate system is to obviate the need for additional system components such as locking nuts and locking rings. This again simplifies manufacture and allows for a more compact overall design.

Further technical advantages of the disclosed locking screw and plate system arise in currently preferred embodiments providing an optional integrated driving handle on the screw unit. In such embodiments, the drive handle is rigidly affixed to the head of the screw. It will be appreciated that the surgeon may require multiple sizes (diameters) of locking screw and plate systems to treat an array of different-sized patients and/or different-sized bone conditions. Providing an integrated driving handle previously affixed to the screw head eliminates the need for multiple drivers or tools to engage the different-sized screws that may be required during fixation.

Likewise, certain screw diameters may be very small—as small as 1 mm or even less. Application of a separate driver or tool to screws of this small size is intricate. Successful torqueing may prove elusive. Providing an integrated driving handle previously affixed to the screw head makes the surgeon's job easier and promotes successful torqueing.

Provision of an integrated driving handle rigidly affixed to the screw head also allows the ratio of driver torque transmission area to screw core cross-sectional area to be engineered to be increased. Increasing this ratio further promotes successful torqueing, especially in the small screw diameter embodiments described in the previous paragraph. Further, if/when the drive handle is “broken off” from the screw head (see paragraph immediately below), provision of shaped screw heads, such as hex or square, enables separate drivers or tools to deliver torque to the screw head once the drive handle is no longer present. Such shaped screw heads may also be engineered to promote increase of the ratio of driver torque transmission area to screw core cross-sectional area.

The rigid connection between driving handle and screw head may further be engineered to fracture (or “break off”) at a desired torque. Optimally, the desired torque will be preselected to be less than the maximum fixation torque for the screw in a particular procedure. The drive handle thus becomes a torque-limiting or torque-controlling tool, eliminating the need for separate such tools during the procedure, further ensuring that the screw is fixed to the bone at the correct torque. This torque-limiting feature of the drive handle also controls the torque for appropriate seating of mating implants such that potential damage does not occur due to over-torqueing.

According to a first aspect, therefore, this disclosure describes embodiment of a screw and plate system, comprising: a screw, the screw having a shaft; a single male thread profile disposed on the shaft, wherein the thread profile is uniform and unitary; a plate, the plate having a thickness; a plate hole provided in the plate; and a female thread profile disposed on an interior of the plate hole, wherein the female thread profile extends through the plate thickness and is configured for matching threaded engagement with the male thread profile on the shaft of the screw; wherein, when the screw is threadably received through the plate hole and into a bone, threaded engagement between the screw and the plate locks the plate in a desired position relative to the bone.

In embodiments according to the first aspect, the screw may be threadably received through the plate hole and into a bone, wherein threaded engagement between the screw and the plate locks the plate in a desired position in contact with an outer surface of the bone.

In embodiments according to the first aspect, the screw may be threadably received through the plate hole and into a bone, wherein threaded engagement between the screw and the plate locks the plate in a desired clearance position above an outer surface of the bone.

In embodiments according to the first aspect, the shaft may have a length from end to end thereof, wherein the single thread profile is disposed along the entire length of the shaft.

In embodiments according to the first aspect, the screw may have a head disposed on one end of the shaft. In other embodiments, the screw and plate system may further comprise an integrated driving handle rigidly connected to one end of the head.

In embodiments according to the first aspect, the screw and plate system may further comprise an integrated driving handle rigidly connected to one end of the screw.

According to a second aspect, this disclosure describes embodiments of a method of orthopedic fixation, the method comprising the steps of: (a) providing a screw and plate system, the screw and plate system comprising: a screw, the screw having a shaft; a single male thread profile disposed on the shaft, wherein the thread profile is uniform and unitary; a plate, the plate having a thickness; a plate hole provided in the plate; and a female thread profile disposed on an interior of the plate hole, wherein the female thread profile extends through the plate thickness and is configured for matching threaded engagement with the male thread profile on the shaft of the screw; (b) preparing a bone to receive the screw; (c) driving the screw through the plate hole via threaded engagement between the screw and plate; and (d) following step (c), driving the screw into the bone to a desired screw depth; wherein, once the desired screw depth is achieved in step (d), threaded engagement between the screw and the plate locks the plate in a desired position relative to the bone.

In embodiments according to the second aspect, the method may further comprise the step of: (e) trimming off a desired portion of the screw once the desired screw depth is achieved in step (d).

In embodiments according to the second aspect, once the desired screw depth is achieved, threaded engagement between the screw and the plate may lock the plate in a desired position in contact with an outer surface of the bone.

In embodiments according to the second aspect, once the desired screw depth is achieved, threaded engagement between the screw and the plate may lock the plate in a desired clearance position above an outer surface of the bone.

In embodiments according to the second aspect, the shaft may have a length from end to end thereof, wherein the single thread profile is disposed along the entire length of the shaft.

In embodiments according to the second aspect, the screw may have a head disposed on one end of the shaft. During step (d), the head may contact the plate when the desired screw depth is achieved. The head may further is trimmed off when the desired screw depth is achieved. Alternatively, the head may be allowed to shear off during step (d).

In embodiments according to the second aspect, the screw and plate system may further comprise an integrated driving handle rigidly connected to one end of the screw to assist with selected ones of steps (c) and (d).

The foregoing has rather broadly outlined some features and technical advantages of the disclosed locking screw and plate system, in order that the following detailed description may be better understood. Additional features and advantages of the disclosed technology may be described. It should be appreciated by those skilled in the art that the conception and the specific embodiments disclosed may be readily utilized as a basis for modifying or designing other structures for carrying out the same inventive purposes of the disclosed technology, and that these equivalent constructions do not depart from the spirit and scope of the technology as described.

### BRIEF DESCRIPTION OF THE DRAWINGS

For a more complete understanding of the embodiments described in this disclosure, and their advantages, reference is made to the following detailed description taken in conjunction with the accompanying drawings, in which:

FIG. 1 depicts various views and features of one embodiment of screw unit 100 configured to interoperate with alternative exemplary embodiments of locking plate units 200 and 300 as shown on FIGS. 2 and 3;

FIG. 1A is an end view as shown on FIG. 1;

FIG. 2 depicts various views and features of locking plate unit 200, a first exemplary embodiment of a locking plate unit within the scope of this disclosure;

FIG. 2A is an enlarged detail as shown on FIG. 2;

FIG. 3 is a section as shown on FIG. 2A;

FIG. 4 depicts various views and features of locking plate unit 300, a second exemplary embodiment of a locking plate unit within the scope of this disclosure;

FIG. 4A is an enlarged detail as shown on FIG. 4;

FIG. 5 is a section as shown on FIG. 4A;

FIGS. 6A and 6B are flow charts describing, in summary, exemplary alternative methods of fixation using embodiments of the locking screw and plate system described in this disclosure;

FIGS. 7 through 13 illustrate various stages of fixation and associated fixation options when using embodiments of the locking screw and plate system described in this disclosure, in which:

FIG. 7 depicts screw unit 100, locking plate 201 and bone 700 ready to begin fixation;

FIG. 8 depicts screw unit 100 threadably received through locking plate unit 200 prior to driving screw unit 100 into bone 700;

FIG. 9 depicts a desired screw depth achieved in optional embodiments in which threaded engagement between screw 102 and locking plate 201 locks the locking plate 201 in a desired position in contact with an outer surface of bone 700;

FIG. 10 is FIG. 9 in which screw head 104 has optionally been removed;

FIG. 11 depicts a stage of fixation towards a desired screw depth in which integrated drive handle 101 has been removed (or has sheared off) and additional torque may be provided by wrench 800 on screw head 104;

FIG. 12 depicts a stage of fixation after FIG. 11 in which a desired screw depth is achieved before screw head 104 reaches locking plate 201, and in which screw head 104 has been optionally trimmed off (or has sheared off); and

FIG. 13 depicts alternative embodiments in which screw unit 100 is substituted for stud 900, with stud 900 driven by chuck tool 850; and

FIG. 14 illustrates an exemplary fixation of bone fracture 702 in which locking plate 201 is locked to bone 700 (but optionally not contacting bone 700) by various exemplary screw embodiments described in this disclosure.

### DETAILED DESCRIPTION

The following description of embodiments provides non-limiting representative examples using Figures, diagrams, schematics, flow charts, etc. with part numbers and other notation to describe features and teachings of different aspects of the disclosed technology in more detail. The embodiments described should be recognized as capable of implementation separately, or in combination, with other embodiments from the description of the embodiments. A person of ordinary skill in the art reviewing the description of embodiments will be capable of learning and understanding the different described aspects of the technology. The description of embodiments should facilitate understanding of the technology to such an extent that other implementations, not specifically covered but within the knowledge of a person of skill in the art having read the description of embodiments, would be understood to be consistent with an application of the disclosed technology.

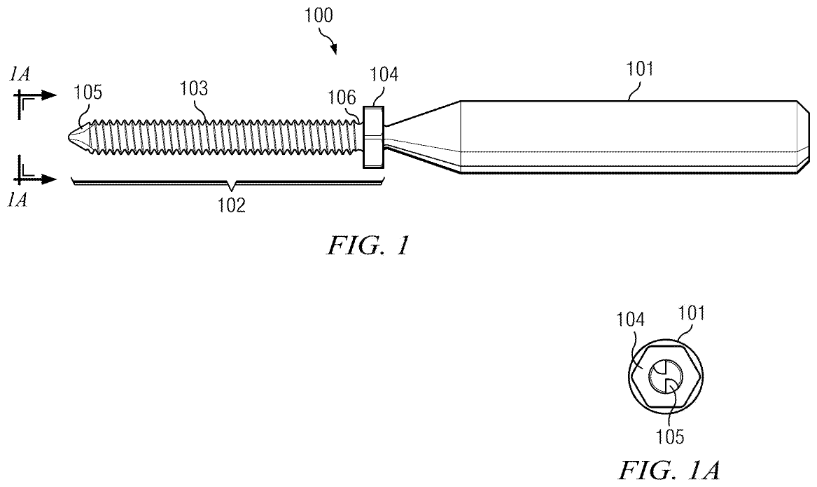

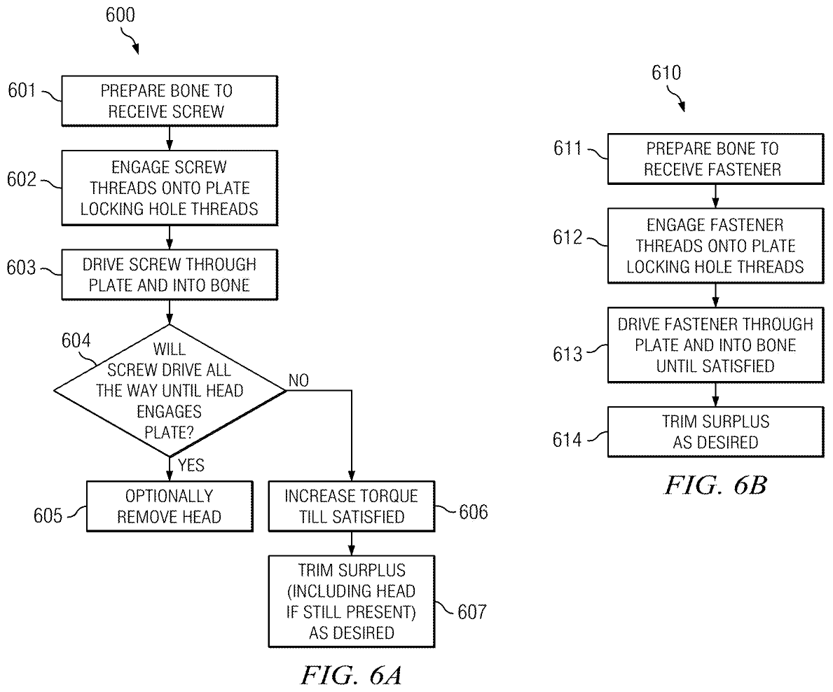

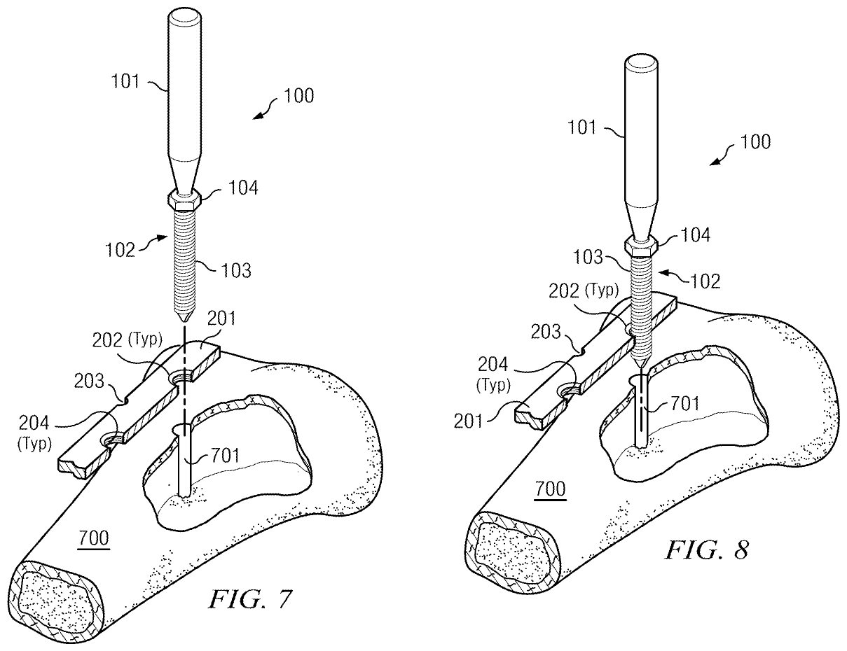

Generally, the Figures in this disclosure should be viewed together as an integrated disclosure. In particular, the following groups of Figures may be viewed together for additional convenience: FIGS. 1, 2 and 3 together; FIGS. 1, 4 and 5 together; FIGS. 6A and 6B together; FIGS. 7 through 14 together; and FIGS. 6A/6B with FIGS. 7 through 14. Depiction or description of assemblies, items, parts or features identified on any one of FIGS. 1 through 14 have the same reference numeral, letter or label where depicted on other Figures or described elsewhere in this disclosure.

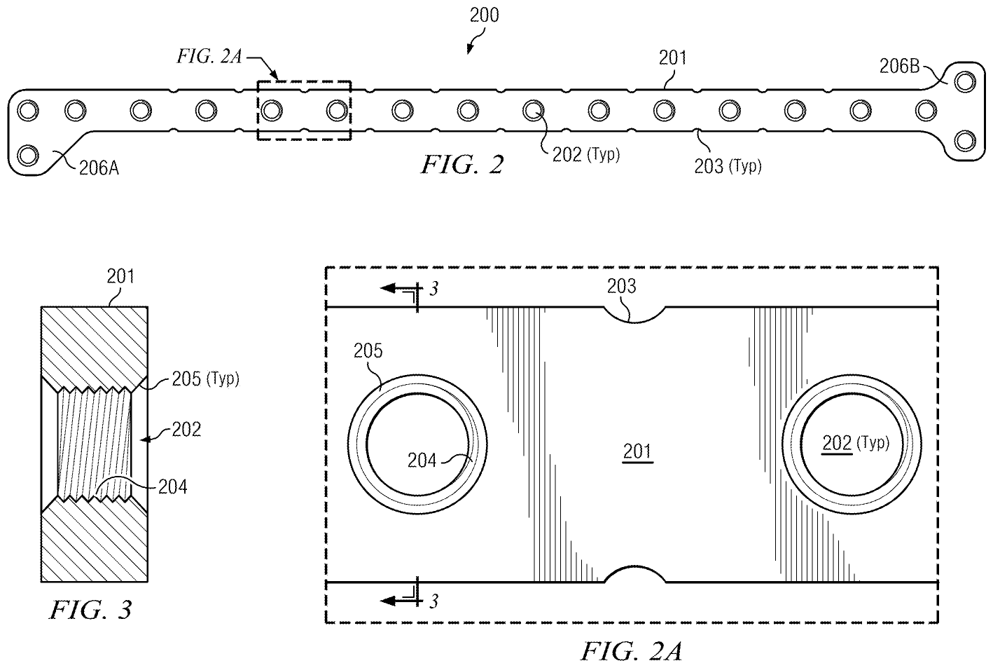

FIG. 1 depicts various views and features of one embodiment of screw unit 100 configured to interoperate with alternative exemplary embodiments of locking plate units 200 and 300 as shown on FIGS. 2 and 3. FIG. 1A is an end view as shown on FIG. 1. Screw unit 100 on FIG. 1 includes screw 102. Screw 102 provides a single, unitary and uniform male thread profile 103, preferably along screw 102's entire shaft length. In some embodiments, and as illustrated on FIG. 1, cutting flutes 105 may be provided at a distal end of screw 102. Advantageously, thread relief 106 is provided between male thread profile 103 and screw head 104. As is conventionally known, thread relief 106 provides clearance for a thread cutting tool during manufacture. A thread relief 106 provided on any particular embodiment of screw 102 is thus intended to be a manufacturing feature, and is expressly not intended to be a thread profile. A thread relief, where provided, should therefore not be interpreted as a thread profile within the scope of this disclosure.

With further reference to FIG. 1, currently preferred embodiments of screw unit 100 include integrated driving handle 101 rigidly connected to screw 102 via screw head 104. Although an optional feature within the overall scope of this disclosure, driving handle 101 nonetheless provides technical advantages to embodiments of screw unit 100 on which it is deployed. These advantages are discussed above in the “Summary” section.

FIG. 2 depicts various views and features of locking plate unit 200. FIG. 2A is an enlarged detail as shown on FIG. 2. FIG. 3 is a section as shown on FIG. 2A. Locking plate unit 200 on FIG. 2 is a first embodiment of a locking plate unit within the scope of this disclosure. Screw unit 100 on FIG. 1 is configured to interoperate with locking plate unit 200 as shown on FIG. 2. Locking plate unit 200 includes locking plate 201, in which at least one (and preferably two or more) locking plate holes 202 are provided. Referring to FIG. 3, each locking plate hole 202 provides a female thread profile 204 disposed on an interior of locking plate hole 202 and extending through the thickness of locking plate 201. Female thread profile 204 is configured for matching threaded engagement with male thread profile 103 on the shaft of screw 102 on screw unit 100 on FIG. 1. Each locking plate hole 202 further has a diameter selected to receive a corresponding screw unit 100 and allow male thread profile 103 on screw 102 on such screw unit 100 to threadably engage with female thread profile 204.

In some embodiments, and as illustrated on FIG. 2, selected locking plate holes 202 may provide countersinks 205 on either side of locking plate 201. Countersinks 205 promote firm contact between screw head 104 and locking plate 201.

In some embodiments, and as illustrated on FIG. 2, cutouts 203 may be provided in locking plate 201 to promote bending flexibility of locking plate 201. In this way, the surgeon may more easily adapt the shape of locking plate unit 200 (“plate contouring”) to suit irregular and curved bone surface profiles during fixation. Such plate contouring advantageously will avoid damaging the integrity of locking plate holes 202 and their female thread profiles 204.

It will be appreciated that in embodiments illustrated on FIG. 2, locking plate 201 is advantageously configured such that screws 102 may enter locking plate holes 202 from either side during fixation. The surgeon is thus not limited as to “which way up” to deploy locking plate unit 200, enhancing convenience, or bringing locking plate shapes and geometries into play whose orientation may be selected according to which side of locking plate unit 200 is facing the bone surface. This feature is illustrated on FIG. 2 by reference to first and second ends 206A and 206B with differing shapes and geometries. The geometry at first end 206A is asymmetric. Now enabled to deploy locking plate unit 200 either way up, the surgeon may flip/rotate/orientate locking plate unit 200 as desired to locate specific end geometries or shapes to suit local bone fixation conditions or requirements.

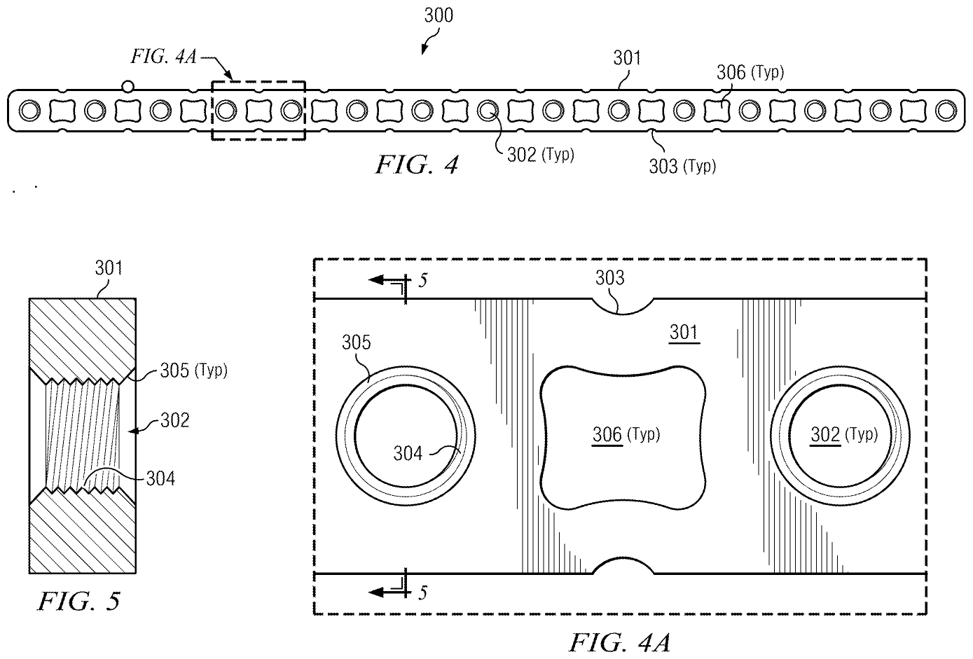

FIG. 4 depicts various views and features of locking plate unit 300. FIG. 4A is an enlarged detail as shown on FIG. 4. FIG. 5 is a section as shown on FIG. 4A. Locking plate unit 300 is a second embodiment of a locking plate unit within the scope of this disclosure. Screw unit 100 on FIG. 1 is configured to interoperate with locking plate unit 300 as shown on FIGS. 4 and 5. Many of the features of locking plate unit 300 on FIGS. 4 and 5 are similar to the corresponding features on FIGS. 2 and 3, and the corresponding description above with reference to FIGS. 2 and 3 applies to locking plate 301, locking plate holes 302, cutouts 303, female thread profiles 304 and countersinks 305 on FIGS. 4 and 5. The main difference between locking plate unit 300 on FIGS. 4 and 5, and locking plate unit 200 on FIGS. 2 and 3, is that in some embodiments, such as illustrated on FIGS. 4 and 5, locking plate unit 300 provides internal cutouts 306. It will be appreciated that in such embodiments, internal cutouts 306 provide yet further bending flexibility to locking plate 301 consistent with description above of cutouts 203 on FIG. 2. Further, internal cutouts 306 (with structural material removed between locking plate holes 302) enable such plate contouring to be performed with reduced potential damage to integrity of locking plate holes 302. Internal cutouts 306 also reduce the overall weight of locking plate unit 300. In certain deployments, internal cutouts 306 may also assist in promoting blood supply to the underlying bone.

In summary, therefore, with reference to embodiments illustrated in FIGS. 1 through 5, this disclosure describes embodiments of a threaded bone screw with a single (unitary), uniform thread along its length and including a head on the plate engaging end of the bone screw. A bone plate is also provided with a threaded hole (holes) corresponding to the thread of the bone screw. The unitary thread form of the screw engages the bone and the bone plate resulting in locking between bone and plate during fixation, with high-functioning angular stability. Once the threaded fastener is advanced such that the screw head is in contact with the plate, further enhanced locking and angular stability is achieved.

With reference now to the Provisional Disclosure, incorporated herein by reference as if fully set forth herein, FIGS. 4 and 5 of the Provisional Disclosure are renditions of FIGS. 1 through 3 herein, in which exemplary physical dimensions are provided for the embodiments of screw unit 100 and locking plate unit 200 illustrated on FIGS. 1 through 3 herein. FIGS. 4 and 5 of the Provisional Disclosure together depict a locking screw and plate system currently available from IMEX Veterinary, Inc. under the VetKISS brand (see IMEX Veterinary's website for more details). It will be appreciated and understood that the physical dimensions set forth on FIGS. 4 and 5 of the Provisional Disclosure are exemplary only for just one embodiment of the locking screw and plate system described in this disclosure, and the scope of this disclosure is not limited in any way to the example depicted in FIGS. 4 and 5 of the Provisional Disclosure.

FIGS. 6A and 6B are flow charts describing, in summary, exemplary alternative methods of fixation using embodiments of the locking screw and plate system described in this disclosure, such as described above with reference to FIGS. 1 through 3. FIG. 6A illustrates a first exemplary fixation method embodiment 600 in which a screw such as screw 102 on FIG. 1 has a head such as screw head 104 on FIG. 1. Fixation on FIG. 6A may be in conjunction with a locking plate such as locking plate unit 200 on FIGS. 2 and 3. A bone is prepared to receive the screw (block 601). In preferred embodiments, a hole is drilled in the bone whose diameter is selected to receive the threads on the screw. However, the scope of this disclosure is not limited in this regard. The screw is then driven through the hole in the locking plate via threaded engagement between the screw and plate (block 602).

The screw is then driven into the bone to a desired screw depth (block 603). In some embodiments, the screw may be driven down to a desired screw depth where the screw head makes contact with the plate. FIG. 6A illustrates this option on blocks 604 and 605. In such embodiments, the screw head may optionally be removed after it contacts the plate (block 605). Threaded engagement between the screw and the plate nonetheless locks the plate in a desired position relative to the bone, providing angular stability between the screw shaft and plate as fixated to the bone even though the head has been removed. Alternatively, the screw head may be left contacting be plate once the desired screw depth has been achieved, in which case the screw head contact with the plate may provide additional angular stability.

In other embodiments on FIG. 6A, the screw may reach a desired depth before the head makes contact with the plate. FIG. 6A illustrates this option on blocks 604, 606 and 607. Increased torque may be needed to achieve a desired screw depth. The screw head may shear off in some deployments where increased torque is applied. Once a desired screw depth has been achieved, however, the surgeon may then optionally trim off a desired portion of the screw, including optionally trimming off the screw head if the screw head is still present (block 607 on FIG. 6A). Such optional trimming may be to any desired height, including flush with the plate. For clarity, such optional trimming of a desired portion of the screw is optional in embodiments where the screw head has not yet made contact with the plate when a desired screw depth is reached, or where the screw head may have already sheared off when a desired screw depth is reached. Threaded engagement between the screw and the plate nonetheless locks the plate in a desired position relative to the bone, providing angular stability between the screw shaft and plate as fixated to the bone even though a desired portion of the screw has been trimmed off once the desired screw depth is achieved.

FIG. 6B illustrates a second exemplary fixation method embodiment 610. Method embodiment 610 is similar to method embodiment 600 on FIG. 6A, except FIG. 6B describes fixation of a fastener on which no screw head is provided (such as a threaded rod or a “stud”). Fixation on FIG. 6B may still be in conjunction with a locking plate such as locking plate unit 200 on FIGS. 2 and 3. Similar to blocks 601 and 602 on FIG. 6A, a bone is prepared to receive the fastener (block 611 on FIG. 6B). The fastener is then driven through the hole in the locking plate via threaded engagement between the fastener and plate (block 612).

The fastener is then driven into the bone to a desired screw depth, or more correctly, a desired fastener depth (block 6B). In distinction to FIG. 6A's description above, however, there are no considerations or options on the fixation method embodiment of FIG. 6B with regard to a screw head since the fastener does not have a screw head. Once a desired screw depth (fastener depth) is achieved, the surgeon may then optionally trim off a desired portion of the fastener (block 614 on FIG. 6B). Such optional trimming may be to any desired height, including flush with the plate. Threaded engagement between the fastener and the plate nonetheless locks the plate in a desired position relative to the bone, providing angular stability between the fastener shaft and plate as fixated to the bone even though a desired portion of the fastener has been trimmed off once the desired screw depth (fastener depth) is achieved.

It will be further appreciated that the fixation method embodiments described above with reference to FIGS. 6A and 6B are indifferent to whether the locking plate makes contact with an outer surface of the bone during fixation. In some embodiments, the locking plate may make contact with an outer surface of the bone when the desired screw depth is achieved. In such contacting embodiments, threaded engagement between the screw and the plate locks the plate in a desired position in contact with an outer surface of the bone. In other, non-contacting embodiments, the locking plate may stand clear of an outer surface of the bone when the desired screw depth is achieved. In such non-contacting embodiments, threaded engagement between the screw and the plate locks the plate in a desired clearance position above an outer surface of the bone. The scope of this disclosure is not limited in either regard.

The scope of this disclosure is also not limited to the manner in which torque is provided to drive the screw through the plate, and on into the bone to a desired screw depth. Non-limiting examples of drive tools for the screw are described in this paragraph. In some embodiments, an integrated drive handle may be provided as described above with reference to FIG. 1. In other embodiments, a wrench may also or alternatively be used to turn the screw head (where a screw head is provided). In fastener embodiments without a screw head, a 3-jaw chuck tool may be used to grip the fastener for rotation. It will be appreciated that gripping the fastener may damage the threads. Optional trimming of the damaged threads may be done once a desired screw depth (fastener depth) has been achieved. Alternatively, in fastener embodiments without a screw head, a driver may be used including a tapped hole on the end for receiving the fastener. Preferably, the tapped hole ends blindly in order to limit the threaded engagement with the fastener, so that further turning of the driver imparts torque to the fastener. The tapped hole driver has the advantage of being simple, and especially suited for small diameter fasteners, even if not as universal as the 3-jaw chuck option described immediately above.

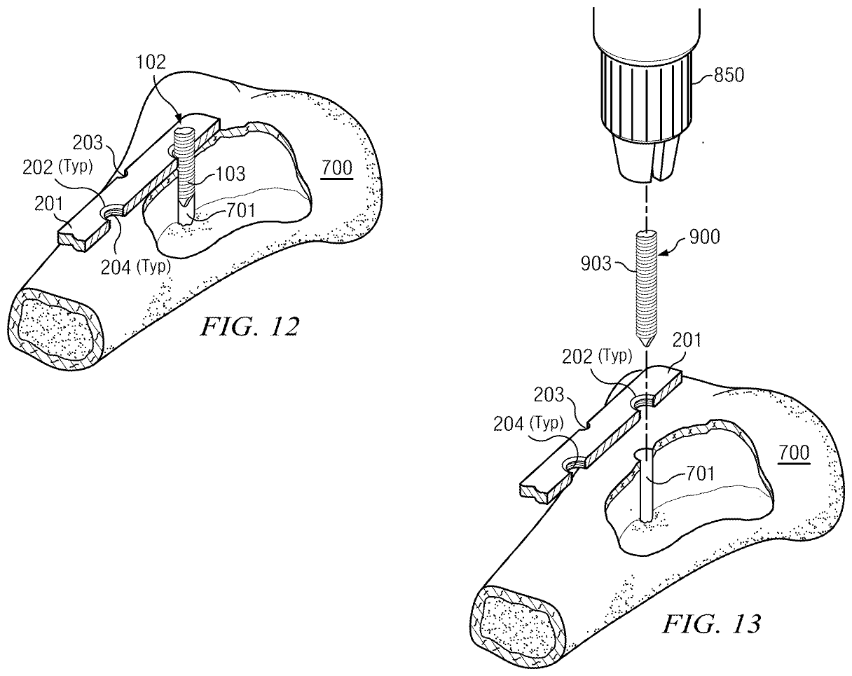

FIGS. 7 through 13 illustrate various stages of fixation and associated fixation options when using embodiments of the locking screw and plate system described in this disclosure. FIG. 7 depicts screw unit 100 from FIG. 1, locking plate 201 from FIGS. 2 and 3, and bone 700, all ready to begin fixation. Bone 700 is prepared to receive screw 102. In the embodiments of FIG. 7 through 13, bone hole 701 is drilled in bone 700. The diameter of bone hole 701 is selected to receive the male thread profile 103 on screw 102 (or the male thread profile 903 on fastener 900 on FIG. 13). FIG. 7 further depicts locking plate hole 202 on locking plate 201 ready to receive screw unit 100.

FIG. 8 is subsequent to FIG. 7, in that FIG. 8 depicts screw unit 100 threadably received through locking plate unit 200 prior to driving screw unit 100 into bone hole 701 in bone 700. Female thread profile 204 on locking plate hole 202 is in matching threaded engagement with the male thread profile 103 on screw 102.

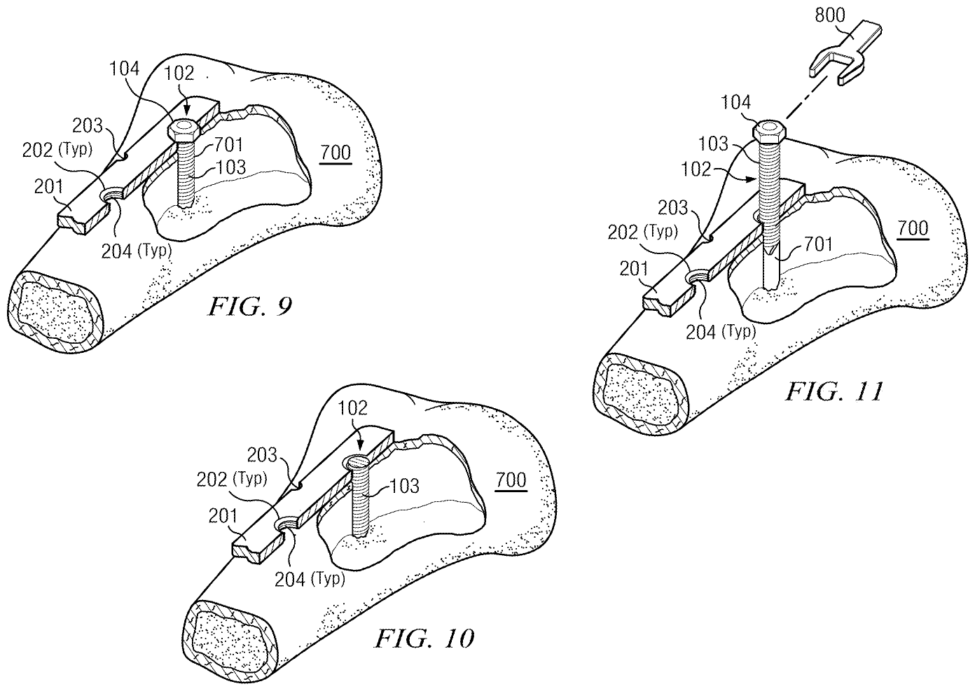

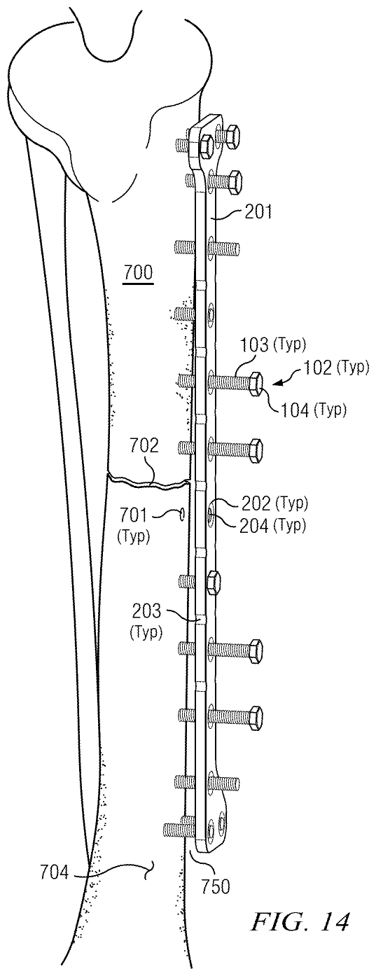

FIG. 9 is subsequent to FIG. 8, in that FIG. 9 depicts a desired screw depth achieved in optional embodiments in which threaded engagement between screw 102 and locking plate 201 locks the locking plate 201 in a desired position in contact with an outer surface of bone 700. As noted above, the scope of this disclosure includes embodiments in which locking plate 201 is in contact with an outer surface of bone 700 (as seen on FIG. 9, for example), and embodiments in which there is a clearance between locking plate 201 an outer surface of bone 700 (as seen on FIG. 14). The scope of this disclosure is not limited in either of these regards.

FIG. 9 further depicts a desired screw depth achieved in optional embodiments in which screw head 104 contacts locking plate 201. Again, as noted above, the scope of this disclosure is not limited in this regard, and other embodiments may provide a desired screw depth achieved where screw head 104 does not contact locking plate 201.

FIG. 10 is subsequent to FIG. 9, in that FIG. 10 depicts where screw head 104 has optionally been removed.

Although related to FIGS. 7 through 10, FIG. 11 depicts a stage of fixation towards a desired screw depth in which integrated drive handle 101 has been removed (or has sheared off) and additional torque may be provided by wrench 800 on screw head 104. Wrench 800 is a hex-shaped flat wrench in the embodiment of FIG. 8, for exemplary engagement with correspondingly-sized screw head 104. However, the scope of this disclosure is not limited in this regard.

FIG. 12 depicts a stage of fixation after FIG. 11 in which a desired screw depth is achieved before screw head 104 reaches locking plate unit 200, and in which screw head 104 has been optionally trimmed off (or has sheared off). The surgeon may then optionally trim off a further desired portion of screw 102, including trimming off screw head 104 if screw head 104 is still present. Depending on the amount of trim desired, fixation at this point may still look like FIG. 12, or may look like FIG. 10. As noted above, trimming is optional, and the scope of this disclosure is not limited to any particular desired amount of trim when trimming is opted for.

FIG. 13 depicts alternative embodiments in which screw unit 100 is substituted for stud 900, with stud 900 driven by chuck tool 850. In the embodiment of FIG. 13, chuck tool 850 is a 3-jaw chuck tool, although the scope of this disclosure is not limited in this regard. As also described above with reference to FIG. 6B, some embodiments described herein may provide fastener embodiments without a screw head such as stud 900 shown on FIG. 9. In such fastener embodiments, chuck tool 850 may optionally be used to grip stud 900 for rotation. It will be appreciated from FIG. 13 that in gripping 900, chuck tool 850 may damage male thread profile 903 on stud 900. Optional rimming of damaged threads on male thread profile 903 may be done once a desired screw depth (fastener depth) has been achieved. Such trimming may be consistent with trimming described above, for example, with reference to FIG. 12.

FIG. 14 illustrates an exemplary fixation of bone fracture 702 in which locking plate 201 as shown on FIGS. 2 and 3 is locked to bone 700 (but optionally not contacting bone 700) by various exemplary screw embodiments described in this disclosure. FIG. 14 illustrates examples of deployment of screws 102 in accordance with options described above with reference to FIGS. 9 through 13, such as (without limitation), screw head 104 optionally left contacting locking plate 201 when a desired screw depth is achieved, screw head 104 optionally removed from screw 102 after screw head 104 contacts locking plate 201 when a desired screw depth is achieved, screw head 104 optionally left on screw 102 but not contacting locking plate 201 when a desired screw depth is achieved, and screws 102 (or studs 900) with a desired portion trimmed off when a desired screw depth is achieved (including trimmed flush with locking plate 201 or trimmed proud of locking plate 201). In each case, it will be understood that threaded engagement between screw 102 (or stud 900) and locking plate 201 locks the locking plate 201 in a desired clearance 750 above an outer surface 704 of bone 700.

It will also be appreciated that the exemplary fixation embodiment illustrated on FIG. 14 is in distinction to the embodiments illustrated on FIGS. 7 through 13 in that, on FIG. 14, locking plate 201 locks the locking plate 201 in a desired clearance 750 above an outer surface 704 of bone 700. By contrast, the embodiments depicted on FIGS. 7 through 13 deploy locking plate 201 making contact with an outer surface 704 of bone 700 when the desired screw depth is achieved. As noted above, the scope of this disclosure is not limited to whether the locking plate 201 makes contact with, or is in clearance from, an outer surface 704 of bone 700. In either case, threaded engagement between the screw 102 (or the stud 900) and the locking plate 201 locks the locking plate 201 in a desired position relative to the bone 700. Angular stability is thus provided between the screw 102 and locking plate 201 as fixated to the bone 700 regardless of whether the locking plate 201 makes contact with, or is in clearance from, an outer surface 704 of bone 700.

Variations:

The following variations, some of which are not illustrated embodiments, are considered within the scope of the locking screw and plate system described in this disclosure:

1. Alternative shapes for screw head (e.g. hex, square, etc.) allow for use of an external fitting driver to be used to tighten or remove screws as needed. Such alternative shapes may be particularly advantageous in embodiments in which a drive handle is originally provided on the screw unit, and after such time that the drive handle has been intentionally “broken off” during fixation (as part of torque control, for example).

2. Round head profile with internal hex or hexolobular recess, for example, for receiving tools to tighten or remove screws.

3. Recessed areas on bone plate to allow screw head to sit lower in the plate reducing profile.

4. Currently preferred embodiments described in this disclosure are advantageously made of implant-grade stainless steel. Other embodiments may be made in whole or in part from other materials, such as, for example, titanium, ceramics or polymer composites. Materials selection may be engineered, for example, (1) at the thread interface between screw and locking plate to provide yet firmer contact and improved angle stability; or (2) in the rigid connection between driver handle (where provided) and screw head to optimize and enhance torque control between the two components.

5. Integrated drive handle engineered to provide pre-determine break point and area from screw. This feature promotes easy handling of screw during fixation. In some embodiments, the driver may break away from the screw as soon as a predetermined locking torque has been reached.

6. An outside hex drive on the screw head may be engineered larger or oversized for the application. This may lead to a better ratio between screw shaft diameter and drive size/diameter/width compared to an inner recess drive. This may also reduce risk of destroying drive during a screw removal operation.

7. Integrated drive handle providing torque sensing to prevent overtightening. This feature may be engineered to maximize the head diameter as connected to an external driver to efficiently transfer torque, particularly if it is desired to remove an implant, for example, (and especially an implant that is overtightened), or alternatively to tighten a screw that is loose. This torque-sensing feature may further minimize instrumentation needed (one size driver may suit many diameters of screws). Further, the torque-sensing feature may obviate any need to incorporate torque limiting devices into drivers not having the torque-sensing feature.

Although the inventive material in this disclosure has been described in detail along with some of its technical advantages, it will be understood that various changes, substitutions and alternations may be made to the detailed embodiments without departing from the broader spirit and scope of such inventive material, some embodiments of which are recited in the appended claims.

We claim:

1. A screw and plate system, comprising:
a screw, the screw having a shaft;
a single male thread profile disposed on the shaft, wherein the thread profile is uniform and unitary;
a plate, the plate having a thickness;
a plate hole provided in the plate; and
a female thread profile disposed on an interior of the plate hole, wherein the female thread profile extends through the plate thickness and is configured for matching threaded engagement with the male thread profile on the shaft of the screw;
wherein, when the screw is threadably received through the plate hole and into a bone, threaded engagement between the screw and the plate locks the plate in a desired position relative to the bone.

2. The screw and plate system of claim 1, in which, when the screw is threadably received through the plate hole and into a bone, threaded engagement between the screw and the plate locks the plate in a desired position in contact with an outer surface of the bone.

3. The screw and plate system of claim 1, in which, when the screw is threadably received through the plate hole and into a bone, threaded engagement between the screw and the plate locks the plate in a desired clearance position above an outer surface of the bone.

4. The screw and plate system of claim 1, in which the shaft has a length from end to end thereof, and in which the single thread profile is disposed along the entire length of the shaft.

5. The screw and plate system of claim 1, in which the screw has a head disposed on one end of the shaft.

6. The screw and plate system of claim 1, further comprising an integrated driving handle rigidly connected to one end of the screw.

7. The screw and plate system of claim 5, further comprising an integrated driving handle rigidly connected to the head.

8. A screw and plate system, comprising:
a screw, the screw having a shaft;
the shaft further having a length from end to end thereof;
a single male thread profile disposed on the shaft, wherein the thread profile is uniform and unitary along the entire length of the shaft;
a plate, the plate having a thickness;
a plate hole provided in the plate; and
a female thread profile disposed on an interior of the plate hole, wherein the female thread profile extends through the plate thickness and is configured for matching threaded engagement with the male thread profile on the shaft of the screw;
wherein, when the screw is threadably received through the plate hole and into a bone, threaded engagement between the screw and the plate locks the plate in a desired position relative to the bone.

9. The screw and plate system of claim 8, in which, when the screw is threadably received through the plate hole and into a bone, threaded engagement between the screw and the plate locks the plate in a desired position in contact with an outer surface of the bone.

10. The screw and plate system of claim 8, in which, when the screw is threadably received through the plate hole and into a bone, threaded engagement between the screw and the plate locks the plate in a desired clearance position above an outer surface of the bone.

11. The screw and plate system of claim 8, in which the screw has a head disposed on one end of the shaft.

12. The screw and plate system of claim 8, further comprising an integrated driving handle rigidly connected to one end of the screw.

13. The screw and plate system of claim 11, further comprising an integrated driving handle rigidly connected to the head.

14. A method of orthopedic fixation, the method comprising the steps of:
(a) providing a screw and plate system, the screw and plate system comprising:
a screw, the screw having a shaft;
a single male thread profile disposed on the shaft, wherein the thread profile is uniform and unitary;
a plate, the plate having a thickness;
a plate hole provided in the plate; and
a female thread profile disposed on an interior of the plate hole, wherein the female thread profile extends through the plate thickness and is configured for matching threaded engagement with the male thread profile on the shaft of the screw;
(b) preparing a bone to receive the screw;
(c) driving the screw through the plate hole via threaded engagement between the screw and plate; and
(d) following step (c), driving the screw into the bone to a desired screw depth;
wherein, once the desired screw depth is achieved in step (d), threaded engagement between the screw and the plate locks the plate in a desired position relative to the bone.

15. The method of claim 14, further comprising the step of;
(e) trimming off a desired portion of the screw once the desired screw depth is achieved in step (d).

16. The method of claim 14, in which, once the desired screw depth is achieved, threaded engagement between the screw and the plate locks the plate in a desired position in contact with an outer surface of the bone.

17. The method of claim 14, in which, once the desired screw depth is achieved, threaded engagement between the screw and the plate locks the plate in a desired clearance position above an outer surface of the bone.

18. The method of claim 14, in which the shaft has a length from end to end thereof, and in which the single thread profile is disposed along the entire length of the shaft.

19. The method of claim 14, in which the screw has a head disposed on one end of the shaft.

20. The method of claim 19, in which, during step (d), the head contacts the plate when the desired screw depth is achieved.

21. The method of claim 19, in which the head is trimmed off when the desired screw depth is achieved.

22. The method of claim 19, in which the head is allowed to shear off during step (d).

23. The method of claim 14, in which the screw and plate system further comprises an integrated driving handle rigidly connected to one end of the screw to assist with selected ones of steps (c) and (d).
